(19) United States
(12) Patent Application Publication
(10) Pub. No.: US 2013/0295024 A1
(43) Pub. Date: Nov. 7, 2013

(54) AEROSOL EMULSIONS

(71) Applicant: James Donald Hammer, Uxbridge, MA (US)

(72) Inventor: James Donald Hammer, Uxbridge, MA (US)

(73) Assignee: Pharmasol Corporation, South Easton, MA (US)

(21) Appl. No.: 13/989,694

(22) PCT Filed: Nov. 18, 2011
(86) PCT No.: PCT/US11/61303

§ 371 Date: Jul. 25, 2013

Related U.S. Application Data
(60) Provisional application No. 61/416,883, filed on Nov. 24, 2010.

(51) Int. Cl. A61K 9/12

(52) U.S. Cl. CPC A61K 9/124
USPC 424/45

(57) ABSTRACT

The present invention relates to water-in-oil aerosol emulsion compositions and related methods and, in particular, to aerosol emulsions containing an active agent. In some embodiments, the aerosol emulsion comprises a propellant dissolved in the oil phase.

### FIELD OF INVENTION

The present invention relates to water-in-oil aerosol emulsion compositions and related methods and, in particular, to aerosol emulsions containing an active agent.

### BACKGROUND

Coatings applied to a skin surface may perform a variety of short term and/or long term functions (i.e., prophylactic and/or drug delivery) and can provide a convenient medium from which to achieve such functionality rapidly. Compositions and methods known in the art include those described in U.S. Pat. No. 4,146,499 (Rosano) and U.S. Pat. No. 4,655,959 (Stopper); U.S. Patent Application Publication No. 2007/0292358; European Patent No. EP0553121 (Neumiller); and International Patent Application Publication No. WO/1994/009642 (Clapp et al.).

### SUMMARY OF THE INVENTION

The present invention relates to water-in-oil aerosol emulsion compositions and related methods and, in particular, to aerosol emulsions containing an active agent. The subject matter of the present invention involves, in some embodiments, interrelated products, alternative solutions to a particular problem, and/or a plurality of different uses of one or more compositions and/or methods.

In one aspect, a composition is provided. The composition comprises a bulk component comprising an aqueous phase comprising a first active agent, a non-aqueous phase comprising a second active agent, wherein the non-aqueous phase is essentially immiscible with the aqueous phase, and a propellant, wherein the propellant is soluble in the non-aqueous phase, wherein the composition is capable of forming a water-in-oil aerosol emulsion.

In another aspect, a composition is provided. The composition comprises a bulk component comprising an aqueous phase essentially free of an alcohol solvent, a non-aqueous phase, wherein the non-aqueous phase is essentially immiscible with the aqueous phase, and a propellant, wherein the propellant is soluble in the non-aqueous phase, wherein the composition is capable of forming a water-in-oil aerosol emulsion.

In still another aspect, a composition is provided. The composition comprises a propellant having a concentration of 20% to 90% by volume in the composition. The composition further comprises a two-phase mixture having a concentration of 10% to 80% by volume in the composition, wherein the two-phase mixture comprises an aqueous phase comprising a first active agent and a non-aqueous phase comprising a second active agent, wherein the non aqueous phase is essentially immiscible with the aqueous phase, wherein the propellant is soluble in the non-aqueous phase.

Other advantages and novel features of the present invention will become apparent from the following detailed description of various non-limiting embodiments of the invention. In cases where the present specification and a document incorporated by reference include conflicting and/or inconsistent disclosure, the present specification shall control.

### DETAILED DESCRIPTION OF THE INVENTION

The present invention relates to water-in-oil aerosol emulsion compositions and related methods and, in particular, to aerosol emulsions containing an active agent. In some embodiments, the aerosol emulsion comprises a propellant dissolved in the oil phase.

In some embodiments, an aerosol emulsion composition comprises a plurality of components. Each component may impart a property to the aerosol emulsion composition. The aerosol emulsion composition may comprise an aqueous phase, a non-aqueous phase (i.e., an oil phase), a bulk component, and a propellant. In some embodiments, the bulk component may comprise an aqueous component and an oil component. In some embodiments, the propellant may be dissolved in the oil component. The aerosol emulsion composition may also include components such as, but not limited to, emulsifiers, active agents, buffering agents, etc., as described in more detail below. It should be understood that one of ordinary skill in the art may include other additives in the aerosol emulsion composition.

Advantageously, water-in-oil aerosol emulsions and related methods can be used in a variety of applications. For example, the compositions and methods may be used for generating drug delivery to a tissue surface, food applications (e.g., butter sprays and cooking oil sprays), protection from damaging sources, antimicrobial and/or antiviral applications, pest repellent, an increase or reduction of friction between two surfaces, and/or ease of cleaning. One particularly advantageous application of the water-in-oil aerosol emulsions is for essentially simulataneous delivery of at least one active agent dissolved in the aqueous phase and at least one active agent dissolved in the non-aqueous phase. Other examples of applications for the aerosol emulsions and related methods are provided below.

As used herein, a “water-in-oil emulsion” refers to an emulsion in which the oil phase (i.e., non-aqueous phase) is the continuous external phase. It should be understood that “aerosol emulsion composition” refers to a composition for generating an aerosol emulsion. It should also be understood that “aerosol emulsion” may refer to airborne particles or to a coating (i.e., film) formed on a surface by the airborne particles.

As discussed above, an aerosol emulsion composition may comprise a propellant and a bulk component. The bulk component may comprise an aqueous phase and a non-aqueous phase (i.e., an oil phase) in a proportion sufficient to form a water-in-oil emulsion. Such proportions can be determined readily by one of ordinary skill in the art.

For example, the aqueous phase in the bulk component may have a concentration of 30 weight % to 70 weight %, in some embodiments, 30 weight % to 60 weight %, in some embodiments, 30 weight % to 50 weight %, in some embodiments, 40 weight % to 70 weight %, in some embodiments, 50 weight % to 70 weight %, and in some embodiments, 40 weight % to 60 weight %.

In some embodiments, the non-aqueous phase (i.e., oil phase) in the bulk component may have a concentration of 30 weight % to 70 weight %, in some embodiments, 30 weight % to 60 weight %, in some embodiments, 30 weight % to 50 weight %, in some embodiments, 40 weight % to 70 weight %, in some embodiments, 50 weight % to 70 weight %, and in some embodiments, 40 weight % to 60 weight %.

In some embodiments, the bulk component in the aerosol emulsion composition may have a concentration of 10 weight % to 80 weight %, in some embodiments, 20 weight % to 80 weight %, in some embodiments, 30 weight % to 80 weight %, in some embodiments, 40 weight % to 80 weight %, in some embodiments, 50 weight % to 80 weight %, in some embodiments, 60 weight % to 80 weight %, in some embodiments, 70 weight % to 80 weight %, in some embodiments, 60 weight % to 70 weight %, in some embodiments, 50 weight % to 70 weight %, and in some embodiments, 60 weight % to 75 weight %.

In some embodiments, the propellant in the aerosol emulsion composition may have a concentration of 10 weight % to 80 weight %, in some embodiments, 10 weight % to 70 weight %, in some embodiments, 10 weight % to 60 weight %, in some embodiments, 10 weight % to 50 weight %, in some embodiments, 20 weight % to 80 weight %, in some embodiments, 20 weight % to 70 weight %, in some embodiments, 20 weight % to 60 weight %, in some embodiments, 20 weight % to 50 weight %, in some embodiments, 20 weight % to 40 weight %, and in some embodiments, 25 weight % to 40 weight %.

In some embodiments, the aqueous phase may comprise water and at least one water-soluble component. For example, in some embodiments, the water phase may contain components such as buffering agents, thickening agents, humectants/moisturizers (e.g., glycerin and propylene glycol), and surfactants (i.e., emulsifiers). In some embodiments, the aerosol emulsion composition is essentially free of an alcohol solvent having fewer than six carbon atoms (e.g., ethanol or isopropanol). In some embodiments, the water phase may be suitable for carrying a water-soluble active agent. In some embodiments, the formulation may include one or more co-solvents. Non-limiting examples of co-solvents include polyols (e.g., glycerin) and alkylene glycols (e.g., propylene glycol).

In some embodiments, the non-aqueous phase (i.e., oil phase) may comprise at least one essentially water-immiscible solvent (i.e., the continuous phase) and at least one oil phase-soluble component. For example, in some embodiments, the oil phase may contain components such as emollients, oils, waxes, esters, and surfactants (i.e., emulsifiers). In some embodiments, the water-immiscible solvent of non-food applications comprises an oil such as lanolin, mineral oil, or silicone oil. In some embodiments, the oil phase may contain other components such as silicone compounds (e.g., high molecular weight silicone fluids and waxes), natural and/or synthetic waxes (e.g., beeswax, carnauba, candelilla, paraffin), petrolatum oil, fatty acids, alcohols, natural oils, synthetic oils, and/or blends thereof. In some embodiments, the water-immiscible solvent may be essentially non-volatile.

In some embodiments, an aerosol emulsion composition may include an emulsifier. In some embodiments, one or more emulsifiers may be selected from groups of low-HLB (hydrophile-lipophile balance) surfactants which are capable of producing a water-in-oil emulsion. Non-limiting examples of emulsifiers include surfactants, glyceryl monostearate, steareth-2, lecithin, polyvinylalcohol, detergents, cetyl alcohol, cetearyl alcohol, polysorbate 20, ceteareth 20, polyoxyl (2) stearyl ether, glyceryl stearate, glyceryl dilaurate, ceteareth-5, oleth-2, similar low-HLB materials, and/or other emulsifiers known to those skilled in the art. In some embodiments, blends of low-HLB and high-HLB materials can also optionally be employed.

In some embodiments, the aerosol emulsion composition may include a thickening agent. Examples of thickening agents include cetyl alcohol, hydroxypropyl cellulose, hydroxypropyl methylcellulose, hydroxypropyl ethylcellulose, hydroxyethyl cellulose, hydroxymethyl cellulose, hydrocolloid gums, guar, acacia, tragacanth, xanthan, locust bean, carrageenan, clay thickeners, magnesium aluminum silicate, sodium magnesium silicate, alumina, bentonite, carbomer, polyox, and/or thickening polymers.

In some embodiments, the aerosol emulsion composition may include a buffering agent. Non-limiting examples of buffering agents include citric acid, lactic acid, benzoic acid, phosphoric acid, EDTA, triethanolamine, and combinations and salts thereof, and other buffering agents known to those of ordinary skill in the art. One of ordinary skill in the art would be able to select a suitable buffering agent to achieve a solution having a pH within a desired range. In some embodiments, the pH of the aqueous phase may be between 3.5 and 8.5, in some embodiments between 4.0 and 8.0, and in some embodiments between 5.0 and 7.0.

The aerosol emulsion compositions may comprise a propellant that is soluble in the non-aqueous phase (i.e., oil phase). Any suitable propellant may be used. For example, the propellant may include gaseous hydrocarbons (e.g., n-butane, isobutane, and propane), halogenated hydrocarbons (e.g., difluoroethane and 1,1,1-chlorodifluoroethane), and mixtures thereof.

In some embodiments, the aerosol emulsion composition may include an active agent. An active agent may be any entity that imparts a desirable property to an aerosol emulsion composition. In some embodiments, the agent may be selected from organic compounds, inorganic compounds, proteins, nucleic acids, and/or carbohydrates.

In some embodiments, the active agent may be dissolved in the aqueous phase. In some embodiments, the active agent may be dissolved in the non-aqueous phase (i.e., oil phase). In some embodiments, the aerosol emulsion composition may include at least one active agent dissolved in the aqueous phase and at least one active agent dissolved in the oil phase. In some embodiments, an active agent may have a solubility of at least 1 g per 10000 mL, in some embodiments, at least 1 g per 1000 mL, in some embodiments, at least 1 g per 100 mL, or in some embodiments, at least 1 g per 10 mL. In some instances, an active agent may have a solubility between 1 g per 10000 mL and 1 g per 100 mL, in some embodiments, between 1 g per 10000 mL and 1 g per 1000 mL, in some embodiments, between 1 g per 1000 mL and 1 g per 10 mL, in some embodiments, between 1 g per 1000 mL and 1 g per 100 mL, in some embodiments, between 1 g per 500 mL and 1 g per 50 mL, in some embodiments, between 1 g per 100 mL and 1 g per 10 mL. Those of ordinary skill in the art would readily be able to determine the solubility of an active agent in the aqueous phase and/or the oil phase.

In some embodiments, the agent may be a pharmaceutical agent. In certain aspects, the pharmaceutical agent may be used to treat the skin. For example, an agent may be an antimicrobial agent (i.e., antiviral, antibacterial, antifungal, etc.), an anti-acne agent, a corticosteroid (i.e., hydrocortisone, clobetasol propionate), nicotine, hormones, anti-inflammatory compounds, external analgesic, anesthetic, antipruritic, counterirritant, antiperspirant, antiseptic, corn/callus remover, antihistamine, antidandruff, first aid antibiotic, pediculicide, skin protectant, skin bleach, sunscreen, and/or wart remover. Other suitable pharmaceutical agents are known to those of ordinary skill in the art.

In some embodiments, an agent may be a skin protectant. Examples of general skin protectants include allantoin, dimethicone, zinc oxide, or zinc acetate. In some embodiments, the skin protectant may protect skin from harmful and/or irritating stimuli. In some embodiments, the skin protectant may provide relief from such stimuli. For example, the skin protectant may reduce pain, iching, or damage. In some embodiments, the skin protectant may protect skin that, for example, has been injured. In some embodiments, the skin protectant may protect exposed skin. In some embodiments, the protection may be temporary.

In other embodiments, the skin protectant may be a sunscreen, such as titanium dioxide, zinc oxide, avobenzone, octocrylene, octylmethoxycinnamate, homosalate, octisalate, or oxybenzone. The term “sunscreen” as used herein includes commonly used ultraviolet ray-blocking compounds such as ethylhexyl p-methoxycinnamate, butyl methoxydibenzoylmethane, 2-hydroxy-4-methoxybenzophenone (benzophenone-3), octyl dimethyl p-aminobenzoic acid, digalloyl trioleate, 2,2-dihydroxy-4-methoxy benzophenone, ethyl 4-[bis(hydroxypropyl)]aminobenzoate, ethyl hexyl p-methoxy cinnamate, 2-ethyl-hexyl salicylate, glyceryl p-aminobenzoate, 3,3,5-trimethylcyclohexyl salicylate, menthyl anthranilate, p-dimethyl aminobenzoate, and 2-ethylhexyl p-dimethyl amino benzoate and the like. Mixtures of these compounds may also be used. The amount of sunscreen component useful in the sunscreen compositions of the present invention is from about 1% to about 30%, with the exact percentage dependent upon the particular agent(s) chosen and SPF level desired. In some embodiments, the sunscreen may be provided in an amount sufficient to provide an SPF rating of at least 10, in certain embodiments at least 15, in certain embodiments at least 20, in certain embodiments at least 25, in certain embodiments at least 30, in certain embodiments at least 45, in certain embodiments at least 60, and in certain embodiments at least 75.

In still other embodiments, an agent may be a cosmetic. One of ordinary skill in the art would readily be able to select suitable agents. For example, an agent may be a hair treatment, moisturizer (e.g., ammonium lactate, butylene glycol, glycerin, hyaluronic acid, propylene glycol, sorbitol, and urea), skin brightener (e.g., sterioids and hydroquinones), skin radiance enhancer (e.g., kiwi fruit extract), anti-aging and anti-wrinkling agents (e.g., retinol, epidermal growth factor, alpha hydroxy acids, beta hydroxy acids, peptides, coenzyme Q10, and anti-oxidants such as vitamin C and vitamin E), skin or hair conditioner (e.g., protein, lanolin oil, olive oil, honey, shea butter, coconut oil, citrus extracts, silicones, humectants, peppermint, lavender, and rosemary), firming/slimming agent (e.g., keratin, wakame kelp, coenzyme Q10, and vitamin E), sunless tanner (e.g., dihydroxyacetone, tyrosine, canthaxanthin, erythrulose, and afamelanotide), coloring agent (i.e., a dye), water-soluble vitamin, herbal extract, and/or fragrance.

In yet further embodiments, an agent may be an insect repellent. Suitable insect repellents include, but are not limited to, DEET, picaridin, plant oils such as citronella, geraniol, lemon eucalyptus, neem, cedar, peppermint, and the like, blends of plant oils, piperonyl butoxide, or pyrethrum.

An agent may also be included that adds or withdraws heat from the skin. For example, spontaneous cooling agents or spontaneous heating agents may be included in an aerosol emulsion composition.

In some embodiments, the aerosol emulsion composition may contain a fragrance. Any suitable fragrance may be used. In some embodiments, a fragrance may be included that can at least partially offset an odor from the aerosol emulsion composition. For example, in some embodiments, a fragrance may obscure an odor from the aerosol emulsion composition such that the odor is essentially imperceptible to a user, i.e., the fragrance may have odor-neutralizing properties. In some embodiments, the fragrance may impart a generally pleasant aroma to the aerosol emulsion composition.

In some embodiments, the active agent may be a food ingredient. For example, the active agent may be a flavor, aroma, vitamin, supplement, non-stick agent (e.g., one or more oils).

An aerosol emulsion composition may contain an active agent at any suitable concentration. A simple test to determine a concentration at which to use an active agent is to prepare a series of aerosol emulsion compositions containing an active agent at various concentrations and aerosolize the compositions. In some embodiments, the aerosol emulsion may be applied to a test surface such as the skin. An assay may be used to monitor the release and/or effectiveness of the active agent over time. For example, in embodiments where the active agent is an antimicrobial, the type and/or quantity of organisms on the test surface may be monitored using techniques known in the art such as microscopy, quantitative PCR, culturing, etc. Based on the results of the assay, the agent concentration may be adjusted in order to achieve the desired effectiveness.

In some embodiments, the concentration of an active agent or other ingredient (e.g., surfactant, emulsifier, thickener, and the like) may be between 0.001% and 30%, between 0.001% and 20%, between 0.001% and 10%, between 0.01% and 30%, between 0.01% and 20%, between 0.01% and 10%, between 0.01% and 5%, between 20% and 30%, between 15% and 25%, between 10% and 20%, between 5% and 15%, between 1% and 10%, between 0.1% and 5%, between 0.01% and 1%, between 0.001% and 1%, or between 0.05% and 2%. It should be understood that concentrations outside these ranges may also be used.

An aerosol emulsion composition may be packaged in a container (e.g., an aerosol can) using any suitable method known in the art. In some embodiments, the water phase and the oil phase may be added to a container before the propellant.

In some embodiments, the aqueous component and the oil component may be phase separated in the container. A water-in-oil emulsion may be generated by any suitable method. For example, the container may be shaken for a period of time sufficient to generate the emulsion.

The water-in-oil emulsion may be stable (i.e., remain as an emulsion) for an extended period of time without further agitation. A stable emulsion is defined as a mixture where at least 95% of the mixture is emulsified. For example, in some embodiments, the water-in-oil emulsion may be stable for at least 5 minutes, at least 10 minutes, at least 20 minutes, at least 1 hour, at least 2 hours, at least 4 hours, at least 8 hours, at least 16 hours, at least 1 day, or at least 2 days.

Aerosol emulsions may be faulted from aerosol emulsion composition having various viscosities. For example, in some embodiments, the viscosity of the aerosol emulsion composition may be at least 1 cP, at least 2 cP, at least 5 cP, at least 10 cP, at least 20 cP, at least 50 cP, at least 100 cP, at least 200 cP, at least 500 cP, at least 1000 cP, or at least 2000 cP. In some embodiments, the viscosity of the aerosol emulsion composition may be between 1 cP and 2000 cP, between 1 cP and 1000 cP, between 1 cP and 100 cP, between 10 cP and 2000 cP, between 10 cP and 1000 cP, between 10 cP and 100 cP, or between 50 cP and 100 cP.

The aerosol emulsion may be used to form a coating (i.e., film) on a surface. The coating may have any suitable thickness. For example, in some embodiments the coating may have a thickness between 1 mm and 100 microns, between 500 microns and 50 microns, between 100 microns and 10 microns, or between 50 microns and 1 micron. The coating may be thicker in some regions as compared to other regions. As a non-limiting example, when used as a skin barrier for a hand, the coating may be thicker on the palm than on the top of the hand (i.e., it may be advantageous to have a thicker coating on regions were more durability may be needed).

In some embodiments, the aerosol emulsion may remain emulsified on a surface for at least 5 minutes, at least 10 minutes, at least 20 minutes, at least 1 hour, or at least 2 hours. In some instances, the emulsion may break, forming a continuous oily coating on the surface.

In some embodiments, the aerosol emulsion coating may be essentially colorless. An essentially colorless aerosol emulsion may be prepared by choosing suitable essentially colorless components. An aerosol emulsion composition may also be essentially transparent to visible light or may be translucent. The transparency or translucency of an aerosol emulsion composition may depend on properties such as the color of the components of the aerosol emulsion composition, the concentration of the components of the aerosol emulsion composition, or the thickness of the aerosol emulsion. These and other properties may be varied by one skilled in the art using routine experimentation until a suitable aerosol emulsion composition is found.

In some embodiments, the coating may remain on the skin for at least 4 hours, at least 8 hours, at least 12 hours, at least 24 hours, at least 36 hours, at least 48 hours, or at least one week. It should be understood that even greater periods of time may be attainable. In some embodiments, the coating may retain one or more properties while on the skin. For example, a coating may retain one or more properties (e.g., antimicrobial properties, antibacterial properties, antifungal properties, insect repelling properties, sun protecting properties, non-stick properties, and the like) while on the surface for one or more of the periods of time listed above. Further description of coatings may be found in U.S. patent application Ser. No. 12/890,187, entitled “Surface Coatings for Skin,” by Hammer, filed Sep. 24, 2010, which is incorporated herein by reference.

Aerosol emulsions of the present invention may be used in any suitable application. For example, the aerosol emulsion composition may have antimicrobial and/or antiviral properties, which may be used in settings such as households, hospitals, clinician offices, food services, schools and daycares, nursing homes, gyms and health clubs, janitorial services, and/or pools or spas. An aerosol emulsion composition may also be used as a skin protectant spray, a first aid for temporary protection of minor cuts, scrapes, burns, etc., and/or a hand sanitizer with residual germ-killing strength. In other examples, the aerosol emulsion may be used as a pain relief spray, an anti-itch spray, or a moisturizing spray. In another example, the aerosol emulsion composition may be used for sun protection. Further still, the aerosol emulsion composition can be used for controlled release of an active agent (i.e., pharmaceutical, prophylactic, and/or cosmetic). In some aspects, the aerosol emulsion composition may be used as a friction barrier, for example to reduce heat generation between two rubbing surfaces.

In some embodiments, the aerosol emulsion may be used in food applications. For example, aerosol emulsion may be used as a non-stick spray (e.g., a butter spray). In some embodiment, the non-stick spray may contain water-soluble milk solids (e.g., dry milk) dissolved in the aqueous phase.

In some aspects, an aerosol emulsion may be used to deliver an agent through the skin. In some embodiments, an agent released from the coating may be absorbed through the skin. In other embodiments, a suitable carrier may be used to facilitate transdermal delivery. Suitable pharmaceutical compositions and methods are described in more detail below. In some embodiments, an agent may be released in a sustained fashion as described in more detail below.

The polymers and particles described herein may be used in “pharmaceutical compositions” or “pharmaceutically acceptable” compositions, which comprise a therapeutically effective amount of one or more active agents associated with an aerosol emulsion composition described herein, formulated together with one or more pharmaceutically acceptable carriers, additives, and/or diluents. The pharmaceutical compositions described herein may be useful for diagnosing, preventing, treating or managing a disease or bodily conditions.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and essentially not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The amount of active agent which can be combined with an aerosol emulsion composition to produce a single dosage form will vary depending upon the host being treated, and the particular mode of administration. The amount of active agent that can be combined with anh aerosol emulsion composition to produce a single dosage form will generally be that amount of the compound which produces a therapeutic effect. Generally, this amount will range from about 1% to about 99% of active ingredient, from about 5% to about 70%, or from about 10% to about 30%.

Examples of suitable aqueous and non-aqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of other materials, such as lecithin, by the maintenance of the required particle size in the case of dispersions, and by the use of surfactants.

The aerosol emulsion composition may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, alkyl benzoates (e.g., C12-C15 alkyl benzoate), propylene glycol, 1,3-butylene glycol, oils (in particular, cottonseed, groundnut, corn, germ, olive, castor and sesame oils), glycerol, tetrahydrofuiyl alcohol, polyethylene glycols and fatty acid esters of sorbitan, and mixtures thereof.

Suspensions, in addition to the coating materials, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

These aerosol emulsion compositions described herein may also contain adjuvants such as preservatives, wetting agents, emulsifying agents, lubricating agents and dispersing agents. Prevention of the action of microorganisms on and/or in the aerosol emulsion compositions may be facilitated by the inclusion of various antibacterial and antifungal agents, for example, paraben, methylparaben, propylparaben, chlorobutanol, phenol sorbic acid, and the like. It may also be desirable to include isotonic agents, such as sugars, sodium chloride, and the like into the aerosol emulsion compositions. In addition, prolonged absorption of the pharmaceutical form may be brought about by the inclusion of agents which delay absorption such as aluminum monostearate and gelatin.

Antimicrobial agents include wide spectrum antimicrobials, narrow spectrum antimicrobials, zinc oxide (i.e., colloidal), silver compounds (i.e., colloidal, ActiCare®, and the like), triclosan, triclocarban, parachlorometaxylenol (PCMX), polyhexamethylene biguanide (PHMB), quaternary ammonium compounds (i.e., benzalkonium chloride, benzethonium chloride, etc.), cetrimonium chloride, domiphen bromide, chlorohexidine gluconate, phenylmercuric salts (i.e., borate, acetate, etc.), phenol-type antimicrobial agents, povidone-iodine, iodophors, parabens, hyantoins, isothiazolinones, iodopropynyl butylcarbamate, 1-(3-chloroallyl)-3,5,7-triaza-1-azoniaadamantane chloride, benzoates, sorbates, propionates, Tea tree oil, and the like.

Antifungal agents include miconazole, tolnaftate, clioquinol, haloprogin, miconazole nitrate, povidone-iodine, tolnaftate, undecylenic acid and related salts (i.e., calcium, copper, zinc, etc.), clotrimazole, and the like.

Delivery systems suitable for use with aerosol emulsion compositions described herein include time-release, delayed release, sustained release, or controlled release delivery systems. Such systems may avoid repeated administrations of the aerosol emulsion and/or active agents in many cases, increasing convenience to the subject and the physician. Many types of release delivery systems are available and known to those of ordinary skill in the art. Specific examples include, but are not limited to, erosional systems in which the composition is contained in a form within a matrix, or diffusional systems in which an active component controls the release rate. The compositions may be as, for example, particles (e.g., microparticles, microspheres), hydrogels, polymeric reservoirs, or combinations thereof. In some embodiments, the system may allow sustained or controlled release of an active agent to occur, for example, through control of the diffusion or erosion/degradation rate of the formulation or particle. The polymers, particles and compositions described herein can also be combined (e.g., contained) with delivery devices such as syringes, catheters, tubes, and implantable devices.

Use of a long-term release coating (i.e., film) may be particularly suitable in some embodiments. “Long-term release,” as used herein, means that the coating is constructed and arranged to deliver therapeutic levels of the composition for at least about 30 or about 45 days, for at least about 60 or about 90 days, or even longer in some embodiments. Long-term release coatings are well known to those of ordinary skill in the art. In some embodiments, a long-term release coating can be formed by coating at least a portion of the skin of a subject, after which the coating remains on the subject for an extended period.

When the aerosol emulsion compositions described herein are administered as pharmaceuticals, to humans and animals, they can be given per se or as a pharmaceutical composition containing, for example, about 0.1% to about 99.5%, about 0.5% to about 90%, or the like, of aerosol emulsion composition in combination with a pharmaceutically acceptable carrier.

The pharmaceutical compositions described herein may be given in dosages, e.g., at the maximum amount while avoiding or minimizing any potentially detrimental side effects. The pharmaceutical compositions can be administered in effective amounts, alone or in a combinations with other compounds. For example, when treating cancer, a pharmaceutical composition may include the aerosol emulsion compositions described herein and a cocktail of other compounds that can be used to treat cancer.

The phrase “therapeutically effective amount” as used herein means that amount of a material or composition comprising an inventive structure which is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of an aerosol emulsion described herein may be from about 10 ng/kg of body weight to about 1000 mg/kg of body weight, and the frequency of administration may range from once a day to a once a month basis, to an as-needed basis. However, other dosage amounts and frequencies also may be used as the invention is not limited in this respect. A subject may be administered one or more aerosol emulsions described herein in an amount effective to treat one or more diseases or bodily conditions described herein.

The effective amounts will depend on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some embodiments, a maximum dose be used, that is, the highest safe dose according to sound medical judgment.

The selected dosage level can also depend upon a variety of factors including the activity of the particular inventive structure employed, the route of administration, the time of administration, the rate of excretion or metabolism of the particular particles or active agents being employed, the duration of the treatment, other drugs, compounds and/or materials used in combination with the particular aerosol emulsion composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

In some embodiments, an aerosol emulsion or pharmaceutical composition described herein is provided to a subject chronically. Chronic treatments include any form of repeated administration for an extended period of time, such as repeated administrations for one or more months, between a month and a year, one or more years, or longer. In many embodiments, a chronic treatment involves administering a coating or pharmaceutical composition repeatedly over the life of the subject. For example, chronic treatments may involve regular administrations, for example one or more times a week, or one or more times a month.

While it is possible for an aerosol emulsion described herein to be administered alone, it may be administered as a pharmaceutical composition as described above. The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition. The kits can further include a description of use of the compositions as discussed herein. Instructions also may be provided for administering the composition by any suitable technique.

The kits described herein may also contain one or more containers (e.g, aerosol cans), which can contain components such as the aqueous component, oil component, propellant, and/or active agent as described herein. The kits also may contain instructions for mixing, diluting, and/or administrating the components. The kits also can include other containers with one or more solvents, surfactants, preservatives, and/or diluents (e.g., normal saline (0.9% NaCl), or 5% dextrose) as well as containers for mixing, diluting or administering the aerosol emulsion compositions to the patient in need of such treatment.

In some embodiments, the compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the particular aerosol emulsion composition and the mode of use or administration. Suitable solvents for compositions are well known and are available in the literature.

The kit, in one set of embodiments, may comprise one or more containers such as vials, tubes, syringes, and the like, each of the containers comprising one or more of the elements to be used in the method. For example, one of the containers may contain the bulk component or propellant. Additionally, the kit may include containers for other components, for example, buffers or diluents to be mixed with the bulk component.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

These above descriptions of applications for the inventive compositions and methods devices are not intended to be exhaustive, and merely illustrate some of the possible embodiments and uses of this invention.

The function and advantage of these and other embodiments of the present invention may be more fully understood from the examples below. The following examples, while illustrative of certain embodiments of the invention, do not exemplify the full scope of the invention. Each of the following examples provide formulations that lack an alcohol solvent. In some embodiments, it may desirable to avoid the use of an alcohol solvent because alcohol solvents can have a de-fatting/drying effect on skin and/or hair, can have toxic effects upon ingestion, and can render a product more combustible or flammable when present in sufficient concentration. Examples 1, 3, and 4 provide formulations that include both an aqueous phase soluble active agent and a non-aqueous phase soluble active agent.

### EXAMPLES

### Example 1

This example provides a skin protectant/pain relief spray formulation according to an embodiment.

| Ingredient | Range (% by wt.) |
|---|---|
| Mineral Oil, USP | 10-20 |
| Cetyl Alcohol | 1-4 |
| Lanolin, USP | 0-15 |
| White Petrolatum, USP | 0-15 |
| Glyceryl Monostearate, NF | 0-4 |
| Steareth-2 | 0-4 |
| Propylparaben, NF | 0.1-0.2 |
| Water | q.s.* to 100.0 |
| Glycerin, USP | 0-15 |
| Pramoxine HCl, USP | 1.0 |
| Phenylephrine HCl, USP | 0.25 |
| Sodium Citrate, NF | 0.0-0.5 |
| Methylparaben, NF | 0.1-0.2 |
| Menthol, USP | 0.0-0.15 |
| BHT | 0-0.05 |

*q.s. = “quantity sufficient”
Bulk 60-75%
Propellant A-46 25-40%

### Example 2

This example provides a skin protectant/pain relief spray formulation according to an embodiment.

| Ingredient | Range (% by wt.) |
|---|---|
| Isopropyl Myristate, NF | 10.0 |
| Cetyl Alcohol, NF | 2.5 |
| Lanolin, USP | 12.5 |
| White Petrolatum, USP | 12.5 |
| Glyceryl Monostearate, NF | 1.0 |
| Propylparaben, NF | 0.1 |
| Water | q.s.* to 100.0 |
| Glycerin, USP | 12.5 |
| Pramoxine HCl, USP | 1.0 |
| Phenylephrine HCl, USP | 0.25 |
| Sodium Citrate, NF | 0.1 |
| Methylparaben, NF | 0.1 |
| Menthol, USP | 0.05 |
| Eucalyptol | 0.1 |
| BHT | 0.05 |

*q.s. = “quantity sufficient”
Bulk 65%
Propellant A-17 35%

### Example 3

This example provides a skin protectant/anti-itch spray formulation according to an embodiment.

| Ingredient | Range (% by wt.) |
|---|---|
| Mineral Oil, USP | 10-20 |
| Cetyl Alcohol | 1-4 |
| Lanolin, USP | 0-15 |
| White Petrolatum, USP | 0-15 |
| Glyceryl Monostearate, NF | 0-4 |
| Steareth-2 | 0-4 |
| Propylparaben, NF | 0.1-0.2 |
| Water | q.s.* to 100.0 |
| Glycerin, USP | 0-15 |
| Hydrocortisone Acetate, USP | 0.5 |
| Sodium Citrate, NF | 0.0-0.5 |
| Methylparaben, NF | 0.2-0.3 |
| BHT | 0-0.05 |

*q.s. = “quantity sufficient”
Bulk 60-75%
Propellant A-46 25-40%

### Example 4

This example provides a skin protectant/anti-itch spray formulation according to an embodiment.

| Ingredient | Range (% by wt.) |
|---|---|
| Mineral Oil, USP | 12.5 |
| Cetyl Alcohol | 2.5 |
| Lanolin, USP | 12.5 |
| White Petrolatum, USP | 12.5 |
| Glyceryl Monostearate, NF | 1.0 |
| Propylparaben, NF | 0.1 |
| Water | q.s.* to 100.0 |
| Glycerin, USP | 12.5 |
| Hydrocortisone Acetate, USP | 0.5 |
| Sodium Citrate, NF | 0.1 |
| Methylparaben, NF | 0.1 |
| BHT | 0.05 |

*q.s. = “quantity sufficient”
Bulk 65%
Propellant A-17 35%

### Example 5

This example provides a moisturizing spray formulation according to an embodiment.

| Ingredient | Range (% by wt.) |
|---|---|
| Isopropyl Myristate | 10-20 |
| Cetyl Alcohol | 1-4 |
| Lanolin, USP | 0-15 |
| White Petrolatum, USP | 0-15 |
| Glyceryl Monostearate, NF | 0-4 |
| Steareth-2 | 0-4 |
| Propylparaben, NF | 0.1-0.2 |
| Water | q.s.* to 100.0 |
| Glycerin, USP | 0-15 |
| Tocopheryl Acetate | 0.1 |
| Retinyl Palmitate | 0.1 |
| Niacinamide | 0.1 |
| Sodium Citrate, NF | 0.0-0.5 |
| Methylparaben, NF | 0.2-0.3 |

*q.s. = “quantity sufficient”
Bulk 60-75%
Propellant A-46 25-40%

### Example 6

This example provides a moisturizing spray formulation according to an embodiment.

| Ingredient | Range (% by wt.) |
|---|---|
| Isopropyl Myristate | 12.5 |
| Cetyl Alcohol | 2.5 |
| Lanolin, USP | 12.5 |
| White Petrolatum, USP | 12.5 |
| Glyceryl Monostearate, NF | 1.0 |
| Propylparaben, NF | 0.1 |
| Water | q.s.* to 100.0 |
| Glycerin, USP | 12.5 |
| Tocopheryl Acetate | 0.1 |
| Retinyl Palmitate | 0.1 |
| Niacinamide | 0.1 |
| Sodium Citrate, NF | 0.1 |
| Methylparaben, NF | 0.1 |

*q.s. = “quantity sufficient”
Bulk 65%
Propellant A-17 35%

### Example 7

This example provides an aerosol butter spray formulation according to an embodiment.

| Ingredient | Range (% by wt.) |
|---|---|
| Canola Oil, USP | 10-50 |
| Mono and di glycerides | 0-4 |
| Butter, NF | 1-25 |
| Polysorbate 80 | 0-4 |
| Glyceryl Monostearate | 0-4 |
| Natural and/or Artificial butter flavor | 0-1 |
| Water | q.s.* to 100.0 |
| Nonfat dry milk | 5-15 |
| Sodium Citrate, NF | 0.0-0.5 |
| Sodium Benzoate, FCC | 0.2-0.3 |
| BHT | 0-0.05 |

*q.s. = “quantity sufficient”
Bulk 60-75%
Propellant A-46 25-40%

### Example 8

This example provides an aerosol butter spray formulation according to an embodiment.

| Ingredient | Range (% by wt.) |
|---|---|
| Canola Oil, USP | 15.0 |
| Mono and di glycerides | 2.0 |
| Butter, NF | 15.0 |
| Polysorbate 80 | 1.0 |
| Glyceryl Monostearate | 1.0 |
| Natural and/or Artificial butter flavor | 0.5 |
| Water | q.s.* to 100.0 |
| Nonfat dry milk | 10.0 |
| Sodium Citrate, NF | 0.1 |
| Sodium Benzoate, FCC | 0.2 |
| BHT | 0.05 |

*q.s. = “quantity sufficient”
Bulk 65%
Propellant A-46 35%

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e., to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed is:

1. A composition, comprising:
a bulk component comprising:
an aqueous phase comprising a first active agent;
a non-aqueous phase comprising a second active agent, wherein the non-aqueous phase is essentially immiscible with the aqueous phase; and
a propellant, wherein the propellant is soluble in the non-aqueous phase, wherein the composition is capable of forming a water-in-oil aerosol emulsion.

2. The composition of claim 1, wherein the aqueous phase has a concentration in the bulk component of 30 weight % to 70 weight %.

3. The composition of claim 1, wherein the non-aqueous phase has a concentration in the bulk component of 30 weight % to 70 weight %.

4. The composition of claim 1, wherein the propellant has a concentration in the composition of 10 weight % to 80 weight %.

5. The composition of claim 1, wherein the aqueous phase is substantially free of an alcohol solvent.

6. The composition of claim 1, further comprising an emulsifier.

7. The composition of claim 1, wherein the composition is capable of forming a water-in-oil emulsion in a container that remains stable for at least 5 minutes.

8. The composition of claim 1, wherein the water-immiscible solvent of the non-aqueous phase is essentially non-volatile.

9. A composition, comprising:
a bulk component comprising:
an aqueous phase essentially free of an alcohol solvent;
a non-aqueous phase, wherein the non-aqueous phase is essentially immiscible with the aqueous phase; and
a propellant, wherein the propellant is soluble in the non-aqueous phase, wherein the composition is capable of forming a water-in-oil aerosol emulsion.

10. The composition of claim 9, wherein the aqueous phase has a concentration in the bulk component of 30 weight % to 70 weight %.

11. The composition of claim 9, wherein the non-aqueous phase has a concentration in the bulk component of 30 weight % to 70 weight %.

12. The composition of claim 9, wherein the propellant has a concentration in the composition of 10 weight % to 80 weight %.

13. The composition of claim 9, wherein the composition is capable of forming a water-in-oil emulsion in a container that remains stable for at least 5 minutes.

14. A composition, comprising:
a propellant having a concentration of 20% to 90% by volume in the composition; and
a two-phase mixture having a concentration of 10% to 80% by volume in the composition, wherein the two-phase mixture comprises:
an aqueous phase comprising a first active agent; and
a non-aqueous phase comprising a second active agent, wherein the non aqueous phase is essentially immiscible with the aqueous phase, wherein the propellant is soluble in the non-aqueous phase.

15. The composition of claim 14, wherein the composition is capable of forming a water-in-oil aerosol emulsion.

16. The composition of claim 14, wherein the composition is capable of forming a water-in-oil emulsion in a container that remains stable for at least 5 minutes.
